(19) United States
(12) Patent Application Publication
(10) Pub. No.: US 2009/0306646 A1
(43) Pub. Date: Dec. 10, 2009

(54) APPARATUS AND METHOD FOR INJECTION ENHANCEMENT OF SELECTIVE HEATING OF A DEPOSIT IN TISSUES IN A BODY

(75) Inventors: Paul F. Turner, Bountiful, UT (US); Mark Hagmann, Salt Lake City, UT (US)

Correspondence Address: THORPE NORTH & WESTERN, LLP., P.O. Box 1219, SANDY, UT, 84091-1219, US

(73) Assignee: BSD MEDICAL CORPORATION, Salt Lake City, UT (US)

(21) Appl. No.: 12/479,670

(22) Filed: Jun. 5, 2009

Related U.S. Application Data
(63) Continuation-in-part of application No. 12/152,513, filed on May 14, 2008.
(60) Provisional application No. 60/930,329, filed on May 14, 2007.

(51) Int. Cl. A61B 18/18

(52) U.S. Cl. 606/33; 977/904

(57) ABSTRACT

An apparatus and method for providing hyperthermia treatments in a deposit surrounded by tissue in a body is disclosed. A radio frequency antenna array is used to direct a radio frequency signal at a selected frequency into the deposit so that the radio frequency signal will have a selected wavelength in the deposit to selectively heat the deposit to a temperature greater than the surrounding tissue through resonant heating within the deposit. An injectable medium is injected in the deposit to increase a dielectric value and/or conductivity value of the deposit. The injectable medium is selected to increase a specific absorption rate of the radio frequency signal within the deposit relative to the surrounding tissue.

### CROSS-REFERENCE TO RELATED APPLICATIONS AND CLAIM OF PRIORITY

This is a continuation-in-part of U.S. patent application Ser. No. 12/152,513, filed on May 14, 2008, which claims priority to U.S. Provisional patent application Ser. No. 60/930,329 filed on May 14, 2007, all of which are herein incorporated by reference in their entirety.

### TECHNICAL FIELD

The present invention generally relates to inducing hyperthermia in a desired target such as cancerous tumor tissue. More particularly, the present invention relates to non-invasively causing localized hyperthermia in a tumor-containing tissue using an antenna positioned outside the tumor-containing tissue.

### BACKGROUND

Certain types of cancerous tumors, such as breast cancer tumors, particularly inflammatory and locally advanced tumors, often resist traditional treatments. It has been statistically shown that sixty to seventy percent of victims of such breast tumors do not survive past five years. The efficacy of conventional methods of treating cancer, such as radiotherapy and chemotherapy, is limited due to necessary constraints on dosage amounts for safety.

For example, it is known that chemotherapy can be applied in sufficient amounts to kill virtually all cancer cells of a tumor. However, the amounts of chemotherapy needed to achieve this can be high enough to cause poisoning of the patient and/or undue side effects. As another example, the intensity of an x-ray beam applied in accordance with radiotherapy cannot be set at an intensity that will damage nearby critical organs and surrounding healthy tissues. Accordingly, there is an ongoing need to develop techniques that enhance existing cancer-related therapeutic procedures so as to increase their effectiveness without increasing the risk of damage to healthy tissue and causing additional discomfort for cancer patients. Breast tumors that have grown to a size of about 3 cm to about 5 cm are particularly hard to treat and are hard to remove surgically, generally requiring removal of the breast to remove the tumor. Alternative treatments for such tumors are needed.

One recent approach toward improving cancer therapy is to subject a tumor to a hyperthermia treatment, i.e., heating of the tumor. The application of heat to cancer cells has been found to increase the efficacy of certain types of therapies for various proposed reasons. Microwave and radio frequency (RF) energy sources have been employed to conduct hyperthermia treatment. Microwave energy has been applied to tumors using waveguides. However, the relatively high frequencies at which microwaves propagate are generally not suitable for deep penetration into tissue.

RF energy at a lower frequency has also been utilized in some instances, and has the potential to achieve greater penetration due to its relatively lower frequencies. However, both microwave and RF techniques have typically used invasive elements, such as wires, catheters, lumens, probes, receivers, and the like. These invasive elements are usually inserted or embedded in the tumor to be treated to ensure proper coupling and focusing of the electromagnetic energy at the tumor site. The use of invasive elements adds complexity to the procedure and is a source of discomfort for patients. Examples of invasive heating techniques using microwave and RF energy are disclosed in U.S. Pat. Nos. 5,928,159; 6,275,738; 6,358,246; 6,391,026; 5,540,737, and 6,468,273.

One prior method for hyperthermia treatment involves the use of phased arrays of dipoles surrounding portions of a body in which a selected portion, such as a tumor, is desired to be heated. The dipoles are operated in a coherent phase or at least a synchronous phase relationship to enable selective targeting of deep tissue tumor masses by controlling the power and relative phase applied to the array of dipoles. These dipoles couple their RF energy to the body through typically deionized water media as it is high in dielectric constant similar to most of the body tissues but is lower in electrical conductivity so it provides small wavelengths but low power absorption. The antenna arrays surrounding such tissue structures have generally been in concentric arrays using lower frequencies with long wavelengths or have been at high frequencies, at or near microwave frequencies, but not in arrangements that would produce selective resonant behavior in tumors of the breast nor create circular polarization that would improve uniformity of such tissue target heating.

Samulski, in U.S. Pat. No. 6,904,323, described the use of phased array dipoles that are place around a cavity containing fluid such as water and where a human breast containing a cancerous tumor can be submerged and heated by the surrounding dipoles antennas. This method uses rather low frequencies that produce a very large wavelength in both high water tissues of the body and in low water content tissues such as mammary fat. The operation, typically at a frequency of about 140 MHz results in a wavelength in fatty tissue of about 86 cm and in muscle tissues is 22.9 cm.

It is desirable to provide a method and apparatus for non-invasively inducing hyperthermia in tumors and malignant tissues that reside within a breast or other protruding portion of a body while avoiding or at least decreasing the potential of excessively heating the surrounding fatty mammary tissue.

### SUMMARY

An apparatus and method for providing hyperthermia treatments to a body portion having tissue surrounding a deposit is disclosed. The apparatus includes a first radio frequency antenna operable to direct a radio frequency signal at a selected frequency into the tissue such that the radio frequency signal will have a selected wavelength to selectively heat the deposit to a temperature greater than the surrounding tissue through resonant heating within the deposit from the radio frequency signal.

The apparatus further comprises an injectable medium operable to be injected into the deposit to increase at least one of a dielectric value and a conductivity value of the deposit. The injectable medium is selected to increase a specific absorption rate of the radio frequency signal within the deposit relative to the surrounding tissue.

### BRIEF DESCRIPTION OF THE DRAWINGS

Additional features and advantages of the invention will be apparent from the detailed description which follows, taken in conjunction with the accompanying drawings, which together illustrate, by way of example, features of the invention; and, wherein:

FIG. 1 is a front schematic illustration of an apparatus in accordance with an embodiment of the present invention;

FIG. 2a is a perspective schematic illustration of an apparatus for heating a spherical deposit contained within tissue in accordance with an embodiment of the present invention;

FIG. 2b is a front schematic illustration of a three dimensional dipole antenna array in accordance with an embodiment of the present invention;

FIG. 2c is an illustration of an embodiment of the three dimensional array of dipole antennas shown in FIG. 2b that is implemented in a system used for hyperthermia treatments in accordance with an embodiment of the present invention;

FIG. 3a illustrates a plot of curves for relative specific absorption rate calculations at a frequency of 915 MHz for typical mammary tissue relative to a spherical deposit contained within the tissue having selected conductivity and dielectric values in accordance with an embodiment of the present invention;

FIG. 3b illustrates a plot of curves for the tissue and deposit of FIG. 3a for various conductivity and dielectric values in accordance with an embodiment of the present invention;

FIG. 3c illustrates a plot of curves for relative specific absorption rate calculations at a frequency of 915 MHz for tissue having a conductivity and dielectric value approximately two thirds that of muscle tissue relative to a deposit having varying conductivity and dielectric values in accordance with an embodiment of the present invention;

FIG. 3d illustrates a plot of curves for relative specific absorption rate calculations at a frequency of 434 MHz for typical mammary tissue relative to a spherical deposit contained within the tissue having selected conductivity and dielectric values in accordance with an embodiment of the present invention;

FIG. 3 e illustrates a plot of curves for relative specific absorption rate calculations at a frequency of 434 MHz for tissue having a conductivity and dielectric value approximately two thirds that of muscle tissue relative to a deposit having varying conductivity and dielectric values in accordance with an embodiment of the present invention;

FIG. 3f illustrates a plot of curves for relative specific absorption rate calculations at a frequency of 100 MHz for tissue having a conductivity and dielectric value approximately two thirds that of muscle tissue relative to a deposit having varying conductivity and dielectric values in accordance with an embodiment of the present invention;

FIG. 3g illustrates additional plots of curves for the tissue of FIG. 3f at the frequency of 100 MHz relative to a deposit having additional conductivity and dielectric values in accordance with an embodiment of the present invention;

FIG. 3h illustrates a plot of curves for relative specific absorption rate calculations at a frequency of 100 MHz for tissue having a conductivity and dielectric value approximately two thirds that of muscle tissue relative to a deposit having a constant dielectric value and varying conductivity values in accordance with an embodiment of the present invention;

FIG. 3i illustrates a plot of curves for relative specific absorption rate calculations at a frequency of 100 MHz for typical mammary tissue relative to a spherical deposit contained within the tissue having selected conductivity and dielectric values in accordance with an embodiment of the present invention;

FIG. 3j illustrates additional curves from FIG. 3i in accordance with an embodiment of the present invention;

FIG. 4 illustrates a schematic diagram of a system having a plurality of electromagnetic radiation applicators powered by an MRI EMR source through a switch in accordance with an embodiment of the present invention;

FIG. 5a is a perspective schematic illustration of a test phantom material configured to simulate a breast having a tumor in accordance with an embodiment of the present invention;

FIG. 5b is a top view of the test phantom material of FIG. 4a that is split in half to measure a temperature of the phantom material in accordance with an embodiment of the present invention;

FIG. 6 is an infrared photo of a phantom breast immediately after being selectively heated, the photo showing the temperature variations for the tumor tissue and normal breast tissue representations;

FIG. 7 is a chart showing a plurality of temperature measurements made across a phantom breast after it has been selectively heated; and

FIG. 8 is a flow chart depicting a method of selectively heating a deposit in a breast in accordance with an embodiment of the present invention.

Reference will now be made to the exemplary embodiments illustrated, and specific language will be used herein to describe the same. It will nevertheless be understood that no limitation of the scope of the invention is thereby intended.

### DETAILED DESCRIPTION OF EXAMPLE EMBODIMENT(S)

Tumors in the breast or other body parts can be selectively heated by directing RF or microwave energy to the body part with a frequency high enough to promote resonant energy absorption in the tumor tissue as compared to the surrounding tissue. Selective heating is particularly successful where the tumor tissue to be heated has relatively high water content compared to the surrounding non-tumor tissue. This enhanced energy absorption relates to a sub-resonant condition behavior of the relatively high water content tissue due to the fact that low water content tissues of the body has rather low electrical conductivity, dielectric constant, and energy absorption with depth as compared to higher water content tissue such as muscle and tumor tissues. The lower dielectric constant results in a longer wavelength in such tissues for all frequencies in the RF and microwave frequency range, with a shorter wavelength in the higher dielectric constant tumor tissue. The higher conductivity tissue can resonate in a relatively small cavity, such as a tumor.

Resonance is a phenomenon that is used to receive and amplify electromagnetic field waves in standard RF and microwave antennas. The antennas are electrical conductors used to enhance electrical currents induced when exposed to electromagnetic fields that have a wavelength that is n times a 1/n ratio of the size of the antenna, where n is a positive integer. The strongest whole object resonance can occur when the wavelength is two times the antenna length for a wire antenna (n=2). The use of resonance to amplify an electromagnetic field has not been reported or studied for tissue structures submerged in the human body. The inventors have discovered that such relationships do occur and can be utilized to provide enhanced and even selective heating of more conductive and higher dielectric constant tissue masses that are surrounded by less electrically conductive and lower dielectric constant tissues.

One aspect of the present invention is the use of this special phenomenon that provides selective heating of a relatively small high water content tumor mass that is surrounded by larger, drier tissues such as mammary tissue or fat. An advantageous application of the present invention is the enhanced level of RF or microwave energy absorption within a tumor of high water content as compared to the surrounding tissues, such as exists in a female breast.

The term radio frequency (RF) generically means electromagnetic radiation with an alternating electric current. It has been common in recent years to define RF extending from frequencies as low as 3 Hz to 300 GHz. The microwave frequency range has been defined to start at various frequencies from as low as 300 MHz for some sources and starting at 1000 MHz for others. Therefore, the terms are not very specific to use. Use of a specific frequency range becomes important when referring to a resonant type of phenomena that leads to selective energy absorption. The term radio frequency or RF will generally be used in this specification, with specific frequencies or frequency ranges of the alternating electric current in the electromagnetic energy disclosed.

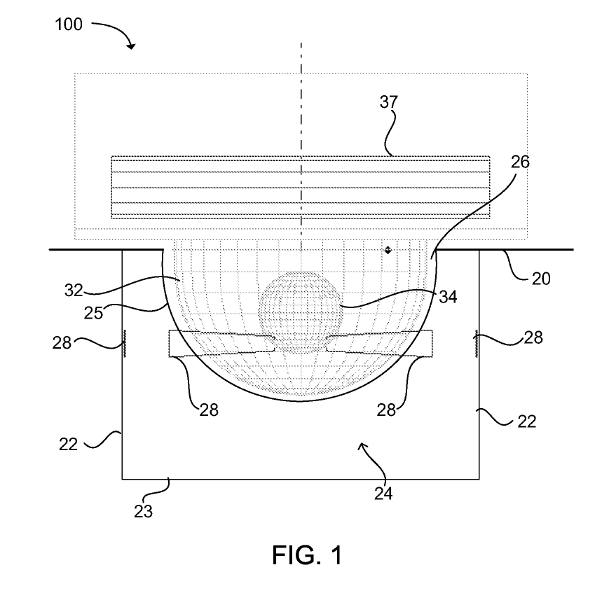

One embodiment of an apparatus 100 for providing hyperthermia treatment for selectively heating tumors located in a protruding portion of a human body is illustrated in FIG. 1. The apparatus comprises an applicator body 25 and a plurality of antennas 28. The applicator body has a concave profile extending from an aperture 26, and defines an open cavity 24 for receiving RF or microwave circularly or elliptically polarized waves. The antennas are operatively associated with the applicator body and are arrayed for transmitting RF or microwave waves at respective selected amplitudes and relative phases into the cavity. The cavity is typically a fluid filled cavity. The electromagnetic energy from the arrayed antennas is generally directed toward a tumor-containing tissue to produce a circularly or elliptically polarized field that will be selectively absorbed by the high water content tissues of a tumor of a size that is partially resonant with the applied fields and their wavelength of the surrounding low water content media.

Without any additional enhancements of the deposit, the amount of resonant energy that is absorbed in a deposit such as a cancerous mass can be optimized by selecting a specific wavelength of electromagnetic radiation that is approximately 3 times greater than a diameter of the mass. However, in most countries, the use of the electromagnetic spectrum is heavily regulated. The radio frequency spectrum has been divided into bands, with each band or frequency typically allowed to be used only for a specific application. The frequency of 915 MHz is an approved frequency for use in hyperthermia treatments in the United States and many other countries.

The wavelength of RF radiation at a frequency of 915 MHz is approximately 14 cm in the fatty tissues of the female breast. This mammary fatty tissue is the dominant tissue of the breast. The wavelength of high water tissues such as muscle material at 915 MHz is approximately 4.36 cm. The table below shows typical values for the RF frequency of 915 MHz for wavelength, penetration, and conductivity in different types of tissue, as published by various sources.

| | | Freq | | Cond. | Wave- | Penetra- |
|---|---|---|---|---|---|---|
| Source | Tissue | (MHz) | Diel. | S/m | length (cm) | tion (cm) |
| Guy | Breast/Fat | 915 | 5.6 | .056-.15 | 13.7 | 17.7 |
| Guy | Muscle | 915 | 51 | 1.60 | 4.46 | 3.04 |
| IFAC | Breast/Fat | 915 | 5.42 | 0.050 | 14 | 25 |
| IFAC | Muscle | 915 | 55 | .948 | 4.35 | 4.2 |
| Gabriel | Breast/Fat | 915 | 5.42 | .050 | 14 | 25 |
| Gabriel | Muscle | 915 | 55 | .948 | 4.35 | 4.2 |

Tissue Value Comparisons of Dielectric Constant

| | Freq | | | | | |
|---|---|---|---|---|---|---|
| Tissue | (MHz) | Guy | IFAC | Gabriel | Joines | Pethig |
| Breast/Fat | 915 | 5.6 | 5.42 | 5.42 | 15 | 5.6 |
| Muscle | 915 | 51 | 55 | 55 | 57.1 | 46 |

Tissue Value Comparisons of Conductivity (S/m)

| | Freq | | | | | |
|---|---|---|---|---|---|---|
| Tissue | (MHz) | Guy | IFAC | Gabriel | Joines | Pethig |
| Breast/Fat | 915 | .056-.15 | .050 | .050 | .18 | .083 |
| Muscle | 915 | 1.60 | .948 | .948 | 1.16 | 1.28 |

The wavelength of RF radiation at a frequency of 120 MHz is approximately 22 to 27 cm in the typical high water content tissues of the human body. At this wavelength, radio frequency energy can be selectively directed deep into the body, including the body center using phased array antennas. For example, a phased array hyperthermia treatment system such as the BSD-2000 available from BSD Medical Corporation may be used to direct RF radiation to a deposit within the body to selectively heat a deposit. As used herein, a deposit is intended to include a tumor, a cyst, or other type of growth that is surrounded by relatively healthy tissue. The selective heating of the deposit can be enhanced through the use of resonant heating. Resonant heating will be described more fully below.

As previously discussed, electromagnetic frequencies having a selected wavelength can resonate within an object of a selected size. When the object is an inner object having a higher conductivity and a higher relative dielectric constant that is contained within a larger object having a lower relative conductivity and a lower relative dielectric constant, the smaller inner object may receive substantially more power from an electromagnetic field due to its size and conductivity.

For example, in a tumor contained within a female breast, the tumor diameter can have a diameter approaching 4.5 cm. This tumor, which has higher water content than the surrounding fatty breast tissue, may therefore experience a self resonant condition leading to increased tumor heating relative to the fatty breast tissue due to the increased resonant currents at a frequency of 915 MHz. With no additional changes made to a tumor, this resonant behavior may also be expected to occur at diameters larger than this but with less intensity.

In another example, in a tumor contained within high water content structures of the body, the optimal resonant tumor diameter can have a diameter approaching 7 to 8.6 cm. This tumor, which has significantly higher water conductivity than the surrounding tissue, may therefore experience a self resonant condition leading to increased tumor heating relative to the surrounding body tissue due to the increased resonant currents at a frequency of 120 MHz. Without additional enhancements to the tumor, this resonant behavior may also be expected to occur at diameters less than or larger than this diameter range, but with a decreased amount of resonance that produces an increase in intensity. So it is reasonable to consider that tumors near this resonant size condition of approximately between 5 to 11 cm in diameter may experience some selective heating at a frequency of 120 MHz due to the resonance of the conductivity enhanced deposit typical of a tumor mass that is surrounded by normal body tissues that contain a relatively high amount of water.

This behavior is similar to a resonant antenna in free space. The phenomena of body resonance for a human body was observed and reported in a U.S. Air Force commissioned study at the University of Utah in 1976, as reported in SAM-TR-76-35 and SAM-TR-78-22 (1978) page 101, which is herein incorporated by reference. In this report, it is shown that a prolate spheroidal model of a chicken egg would be of major axis size 5.8 cm and minor size diameter of 4.4 cm. When the chicken egg is located in an air medium and exposed to a 2 GHz field it becomes resonant with the field. On resonance in an electromagnetic field having an intensity of 1 milliwatt per centimeter squared (mW/cm²), the specific absorption rate (SAR) of the egg is between 0.35 to 0.46 Watts/kg. At higher frequencies, where the egg diameter is no longer resonant due to the shorter wavelength of the RF signal, the absorption drops to about 0.11 W/kg.

The free space wavelength of an RF signal at a frequency of 2 GHz is 15 cm. So the resonant diameter size of the spheroid object is between 0.293 to 0.386 times the wavelength, or in other words approximately one fourth to one third of the wavelength. The free space wavelength of an RF signal at a frequency of 915 MHz is approximately 32.8 cm. The spherical resonance size for 915 MHz when surrounded by free space would be expected to be between 9.6 cm to 13.0 cm. This is calculated based on simple frequency scaling. The spherical resonance size for 120 MHz when surrounded by free space is between 73.0 cm to 99.0 cm.

For the condition that a spheroid, such as a cancerous tumor having a higher conductivity and dielectric value than the surrounding tissue, is contained within a uniform media such as fatty breast tissue that has a dielectric constant greater than free space, the resonant conditions can occur for a smaller spheroid that is scaled by the ratio of the wavelength differences. For example, the wavelength of the fatty breast tissue for a 915 MHz signal is approximately 14 cm. The same RF signal in free space is approximately 32.8 cm. Thus, the ratio of the wavelength of the signal in the breast tissue relative to the signal in free space is approximately 0.427.

Based on this ratio, the size of such an unaltered spheroid tumor in a fatty mammary tissue region would be between 4.1 cm to 5.5 cm to enable resonance to occur for an electromagnetic signal having a frequency of 915 MHz. The resonance behavior was shown in the Air Force reports to be rather broad. An increased specific absorption rate of approximately four times can occur in a resonant body based on the increase in absorption of the electromagnetic field due to the resonant dimensions of the body relative to the wavelength of the signal in the body. The frequency bandwidth to half the peak value of the resonant phenomena was shown to be about two times the resonant frequency. Therefore, it would be expected for an RF signal at a frequency of 915 MHz that the size range of a resonant body, having an unaltered conductivity or dielectric value, which would at least double the SAR relative to an outer body, could range from a size of 2.7 to 11 cm. This would cover the range of most primary advanced breast cancerous tumors.

Further scaling of the curve disclosed on page 101 of the SAM report shows that when an object is much smaller than the resonant size the SAR in the smaller body drops off very rapidly with frequency when no alterations are made to the object's conductivity or dielectric value. For example, a spheroid diameter that is 1/5.5 in size relative to the optimum resonant diameter has a SAR that is approximately one fifth the absorption value of the body and only 5% of the SAR at resonance. So a tumor at 5.5 cm in size on resonance at 915 MHz that had a 4 to 5 times increase in SAR due to resonance would have a SAR 20 times greater than that of a 1 cm diameter tumor. Therefore, this unique resonant condition would not favor small tumor heating in the breast at 915 MHz under these modeled conditions when no alterations are made to the tumor's conductivity and dielectric values.

Although the breast is internally dominated by mammary fatty tissues, there are glandular, ductal and lobular networks that may provide very small but higher conductivity and dielectric pathways for these radio frequency currents to flow and concentrate. However, these networks do not dominate the tissue construction in the breast and therefore do not significantly alter the resonant behavior and conditions. Tissue structures such as ductal and lobular networks however may themselves have selective increased SAR due to their higher conductivity, thereby causing selective pathways for the radio frequency currents within the breast. Also, since the breast mammary tissues themselves are more conductive than free space, it should be expected that the resonant enhancement of the specific absorption rate in a tumor relative to the absorption rate of the surrounding breast material may not be as great as shown in the SAM report of the egg relative to air. The surrounding mammary tissue may lower the resonant behavior similar to resistive loading of a resonant electrical circuit.

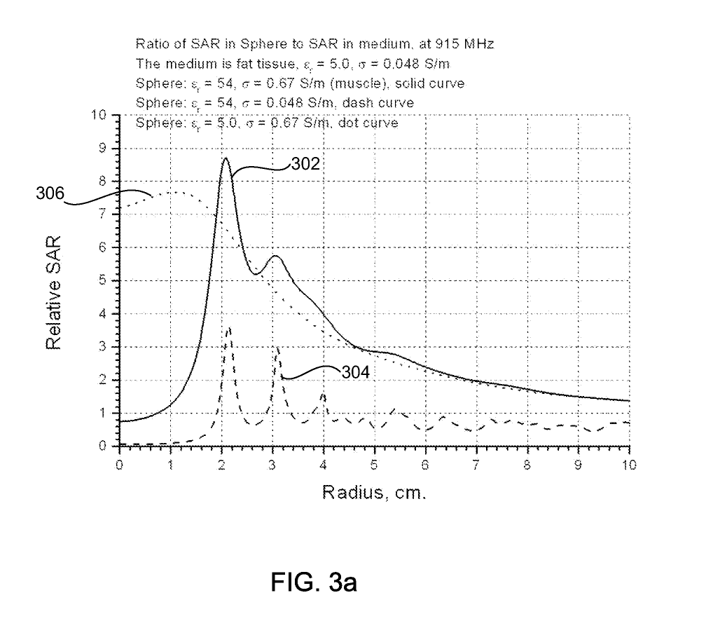

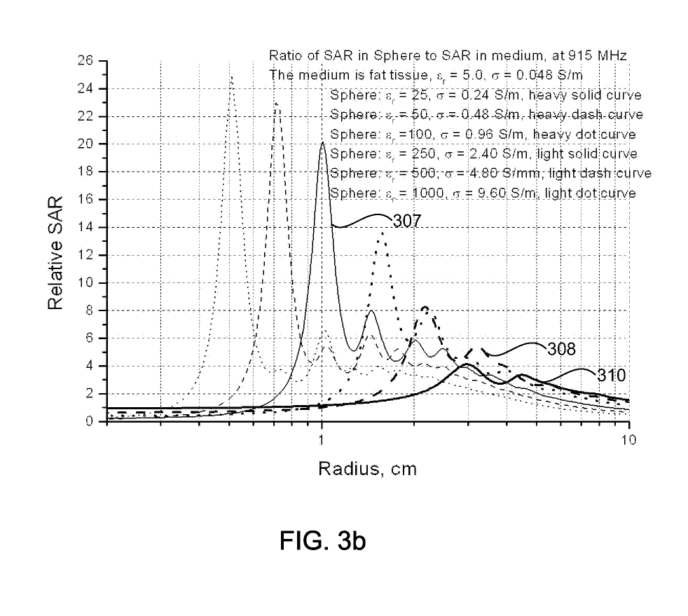

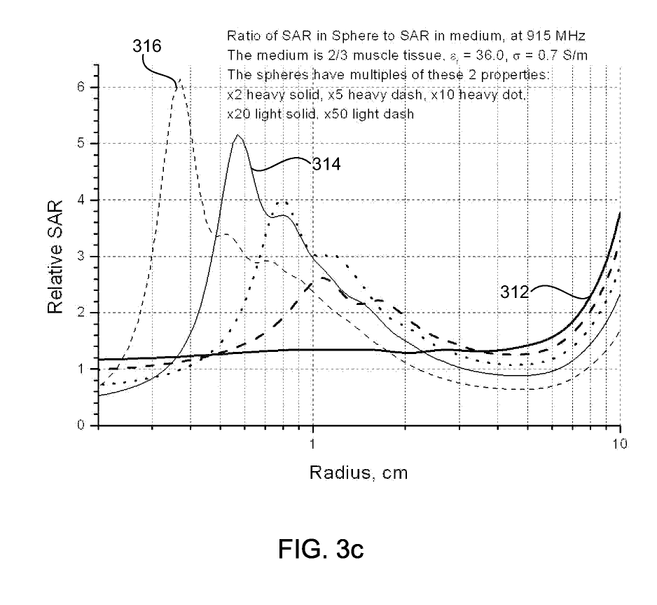

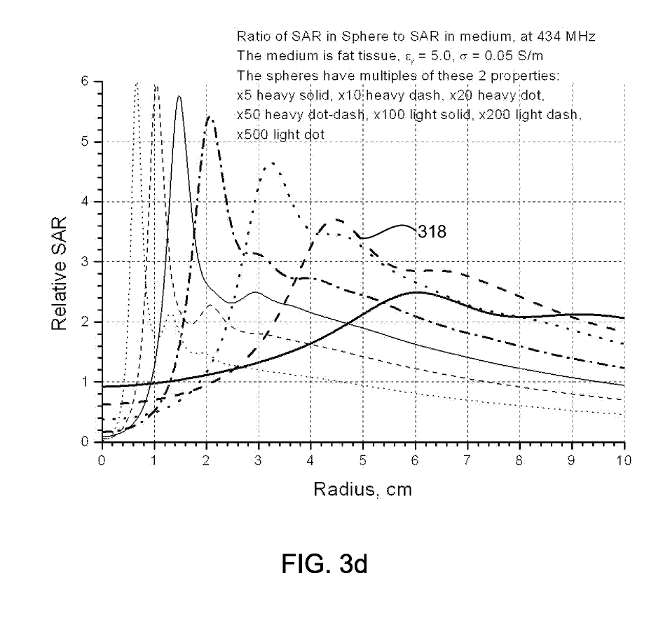

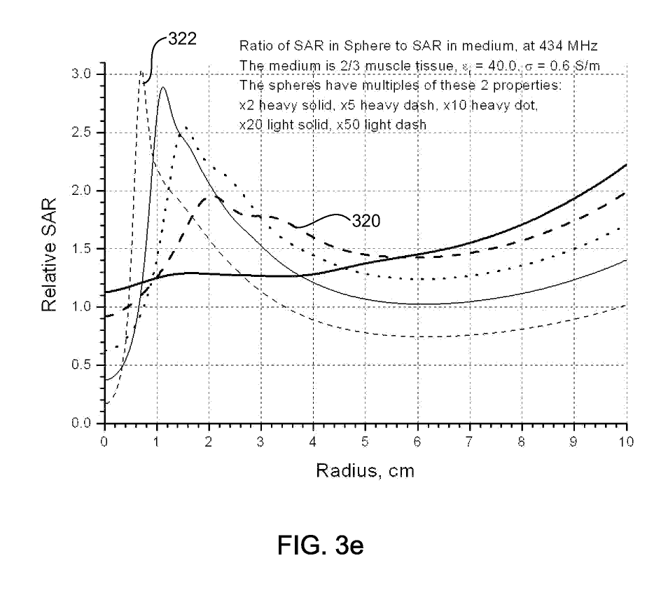

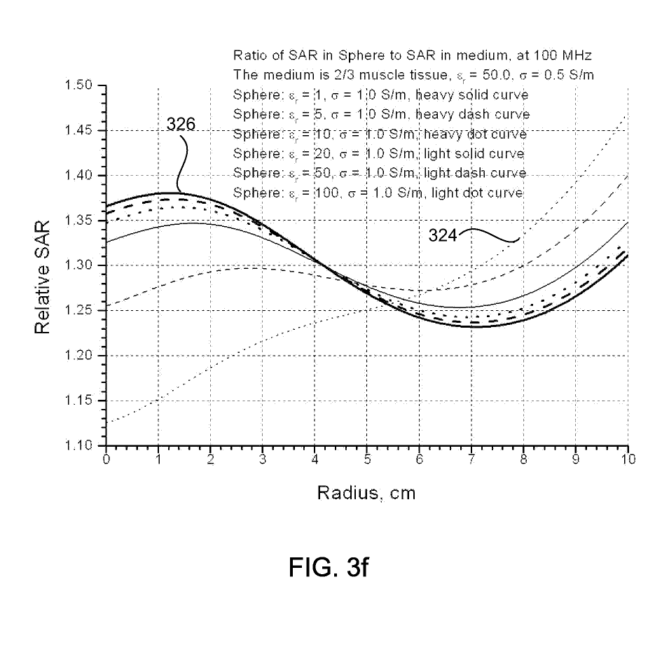

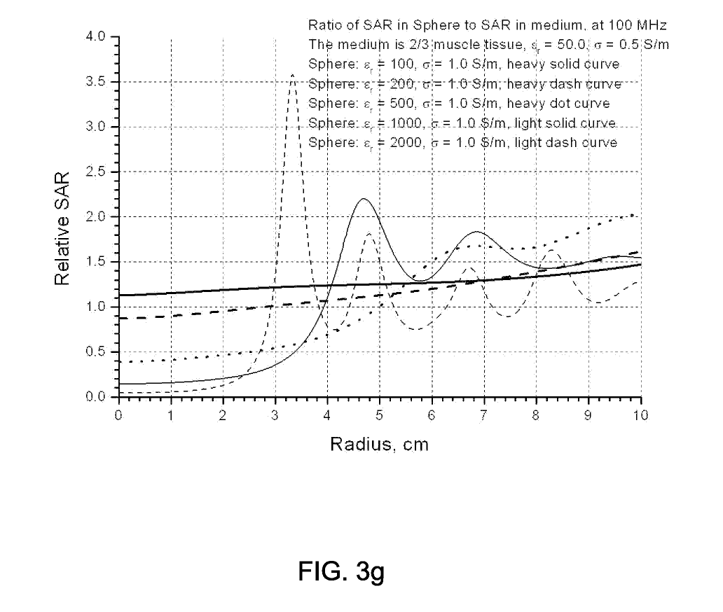

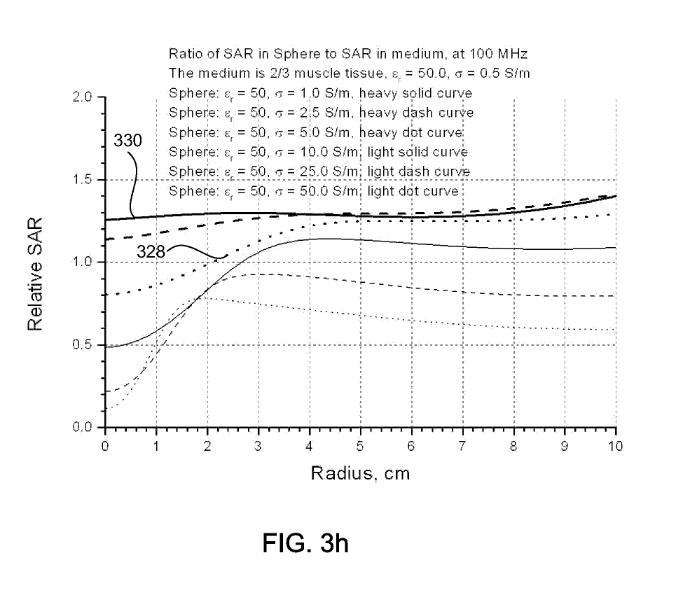

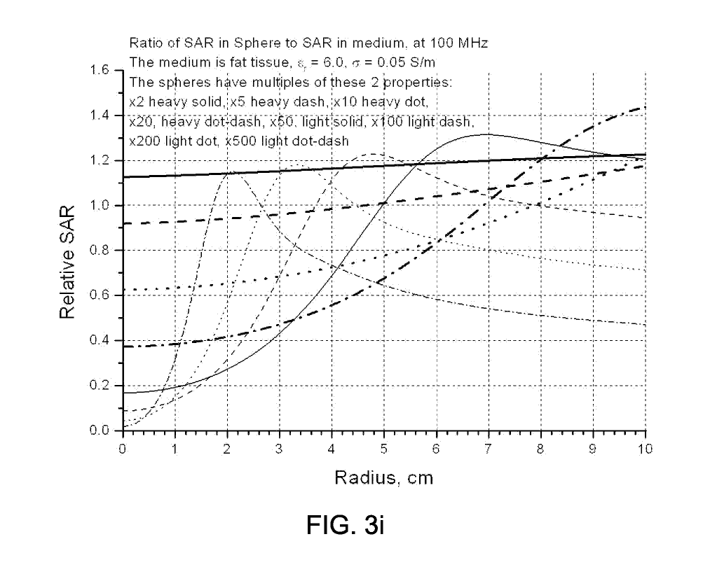

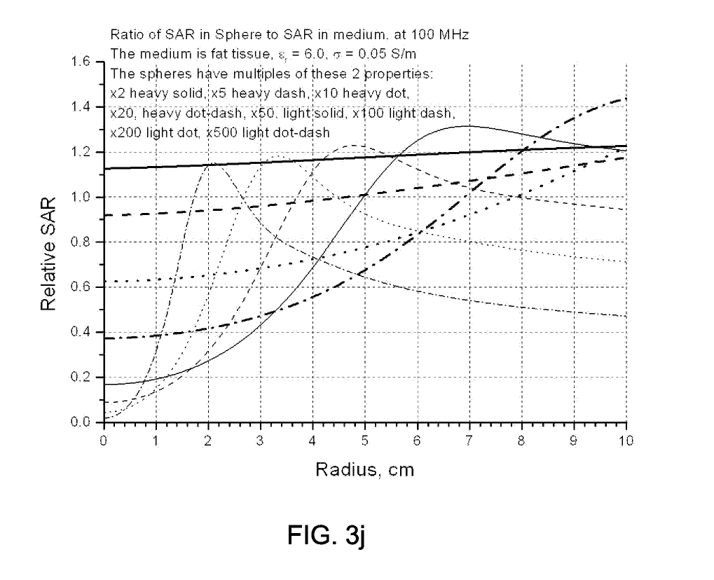

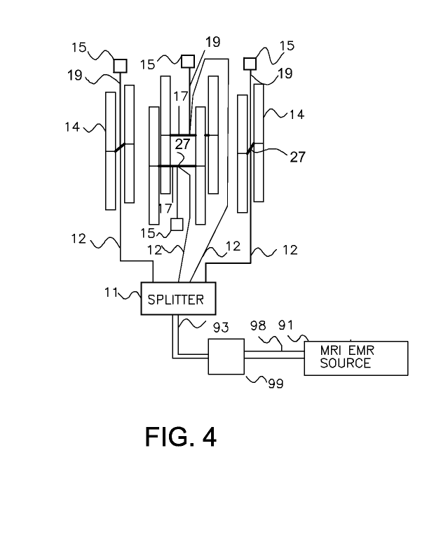

The specific use of the 915 MHz frequency band that has a wavelength in mammary tissue of about 14 cm then can have the capability to excite a resonance in a higher water tissue such as a malignant tumor that has a diameter of approximately 4.5 cm. Note that even though a 3.5 cm diameter tumor does not exactly meet this criteria for the most selective tumor absorption, such a diameter at those conditions is in a state to have some resonance enhancement, but the greatest enhancement would be in a tumor with a diameter that is about ⅓^{rd} of the wavelength in the mammary tissue. Fortunately, most advanced tumors of the breast that become difficult for successful surgical removal are those that exceed about 3 cm in diameter. Thus, this resonant phenomenon, when properly created in these larger tumors, may provide a desirable option in treating such cancerous tumors of the breast. Additional enhancements to the phenomena can be achieved by selectively increasing tumor conductivity and dielectric values through injection of conductivity and dielectric enhancing media such as saline solution and nanoparticles. Nanoparticles capable of being injected into a tumor and distributed through tumor cells are currently being developed for sale in the marketplace. The use of nanoparticles will be discussed in more detail with respect to FIGS. 3a-3j and 4a-4b.

A linearly polarized electromagnetic signal can be used to induce a resonant behavior in a tumor. Such is generally the optimal approach for devices such as the BSD-2000 system that uses deep phased array of dipoles surrounding the body. For deep body target deposits, a linearly polarized antenna array may be preferred to selectively heat a conductivity enhanced deposit target such as a cancerous tumor.

However, the use of linear polarization will tend to have a less uniform distribution of power within the tumor and even the mammary tissue and may result in undesirable hot spots in normal mammary tissue or cooler zones in the tumor. The use of elliptically or circularly polarized electromagnetic radiation to induce resonant heating in a tumor can provide more even heating.

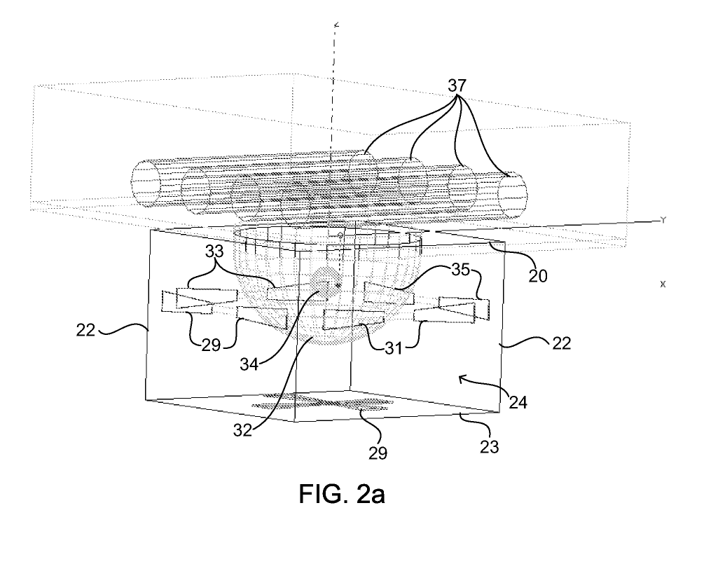

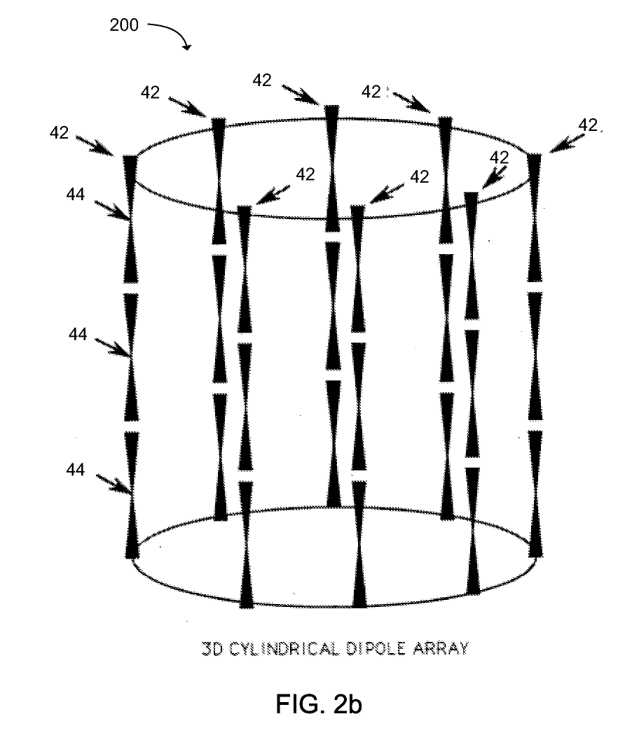

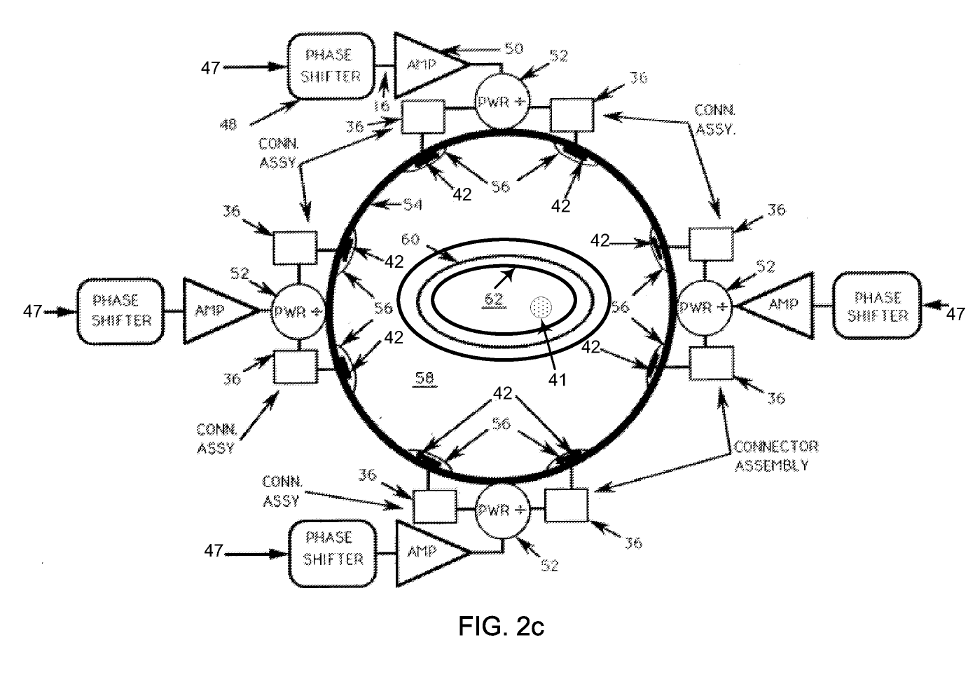

Referring to FIGS. 1 and 2a, an apparatus in one embodiment of the present invention includes an applicator body with a top peripheral surface 20, and side walls 22 with bottom wall 23 forming a cavity 24 which depends from an opening 26 in surface 20. An RF or microwave dipole antenna 28, shown as a bow tie dipole antenna, is positioned on each of the cavity walls 22 to form an antenna array surrounding the cavity 24. Additional dipole antennas 29 may be provided on bottom wall 23 as part of the antenna array. In one embodiment, the applicator can be a portable unit with the peripheral surface 20 merely forming a top surface for the applicator.

In another embodiment, the applicator can be built into a larger unit with a patient support surface 20 being a relatively comfortable supporting surface. The patient can lie on the support surface in a position so that the protruding body part of the patient can be extended into and be received in cavity 24.

In the embodiment illustrated, a female patient having breast cancer with a breast tumor is positioned with the breast 32 to be treated extending into cavity 24. The breast tumor 34 is illustrated as substantially centered in the breast 32. Patient ribs are schematically represented as 37. Cavity 24 may be filled with a dielectric fluid to improve transmission of the RF or microwave energy to the breast. While the dielectric constant of the fluid is not critical, it has been found that fluids with a dielectric constant between about two and about eighty-one may be used satisfactorily. Water, with a dielectric constant of about seventy-eight, can be used. Oils, such as mineral oil or other oils with dielectric constants of about two to about four, vegetable oils, liquid silicones, or other fluids, such as for example propylene glycol or ethylene glycol, with dielectric constants between that of oils and water can also be used. A dielectric fluid that is substantially non-ionic and has a relatively low dielectric constant can minimize heating within the fluid, thereby allowing for greater cooling at a surface of the breast tissue 34.

While the dielectric fluid(s) can be placed in the cavity 24 and come in direct contact with a breast or other body part placed in the cavity, it is usually preferred to provide a thin plastic or rubber membrane 25 in cavity 24 that separates the breast from the dielectric fluid provided in the bolus. The bolus membrane 25 can be formed by, for example, a silicone, urethane, or similar flexible membrane or film to prevent direct contact between the dielectric fluid and the body part. This protects the patient from contact with the fluid.

The dielectric fluid can also be used to control heating of the surface of the breast. The dielectric fluid may be at a lower relative temperature. Contact between the breast 32, or other body part, and the bolus membrane 25 can be used to transfer excess heat from the outer tissue to the dielectric fluid. In one embodiment, the dielectric fluid in the cavity 24 may be circulated and cooled to provide surface cooling for the breast or other body part received in the cavity.

The size of the cavity 24 can vary. The cavity will typically be kept relatively close to the size of the body part to be received. Where a breast is to be treated, the perimeter of the cavity 24 to receive the breast works well when no more than about one and one half times the size of the base of the breast. A cavity with fifteen centimeter side walls has been found generally satisfactory for use with most breasts.

The applicator antennas 28 are connected in typical manner to a radio frequency signal source. The applicator antennas used can be of various types such as spiral antennas, waveguides, helical antennas, Tee Dipoles, and other common applicators used to radiate radio frequency radiation to heat tissue. The frequency and power output from the signal source can be controlled to provide a wavelength within the fatty mammary tissue where the diameter of the tumor is approximately ⅓^{rd} of the wavelength in the fatty tissue. For example, if the frequency of the RF signal supplied to the antennas is 915 MHz, that frequency will produce a wavelength in the normal fatty breast tissue of about 14 cm. This wavelength in the fatty mammary breast tissue will then have the capability to excite a resonance in higher water tissue, such as in malignant tumor tissue, when the diameter of the malignant tumor tissue is approximately 4.5 cm. If the tumor to be treated has a diameter of about 4.5 cm, the tumor may exhibit full resonance excitement behavior and be selectively heated by the applied electromagnetic signal relative to the fatty breast tissue.

While it has been found that full resonance behavior occurs when the tumor diameter is approximately ⅓^{rd} of the wavelength in the fatty tissue, it has also been found that significant resonant behavior is exhibited within about plus or minus twenty-five percent of the one-third dimension. Thus, although a 3.5 cm diameter tumor does not exactly meet the one-third diameter criteria for the most selective tumor absorption, such a diameter still is in a state to have some resonance enhancement, and show significant selective heating over the fatty normal breast tissue.

Since, as indicated, most advanced tumors of the breast that become difficult for successful surgical removal are those that exceed about 3 cm diameter size, a radio frequency of 915 MHz will produce significant selective heating of such tumors in a breast. These resonant phenomena, when properly created in these relatively larger tumors, can provide a desirable option in treating such cancerous tumors of the breast. Where allowed, the frequency of the applied RF energy can be adjusted to provide substantially a one-third ratio between the tumor diameter and the wavelength in the normal fatty breast tissue. The desired wavelength to be produced in the surrounding tissue to provide substantially maximum resonant phenomena in the tumor is determined by multiplying the diameter of the tumor by π (3.14).

In one embodiment, a single antenna 28 can be used to direct radio frequency waves at a selected frequency into the breast 32 and tumor 34, as illustrated in FIG. 1. However, emitting the radio frequency waves from a single antenna will provide more heat at a side of the breast relative to the location of the antenna. To provide more even heat, a second antenna 35 located opposite the first antenna 29 can be used, as illustrated in FIG. 2a. The radio frequency waves from the two antennas can be emitted in phase, thereby allowing the radio frequency waves to interfere within the cavity area 24. The two oppositely located antennas can provide heating to both a front and a back of the tumor. Additionally, an interference pattern may form, resulting in relatively hot and cold areas across the breast and tumor. Uneven absorption and reflection of the waves can also result in additional uneven heating of the tumor.

To reduce uneven heating within the cavity 24, an antenna 31 can be located with an emitting axis that is orthogonal to the emitting axis of another antenna 29. The orthogonal antennas can be tuned to be approximately 90 degrees out of phase relative to the other antenna. The resulting output from the two antennas is a substantially circularly polarized electromagnetic field within the cavity area. A total of four antennas 29, 31, 33 and 35 can be used to produce circularly polarized electromagnetic fields that substantially surround the breast. The circular polarization can effectively stir the electromagnetic fields within the chamber, thereby reducing and eliminating hot spots that can develop and occur within the heating process. If the orthogonal antennas are less than or greater than 90 degrees out of phase, the result will be elliptically polarized electromagnetic fields that can also be used to reduce and eliminate hot spots and cool spots to provide more even heat distribution within the breast 32 and tumor 34.

In another embodiment, shown in FIG. 2b, a three dimensional array 200 of dipole antennas 42 can be constructed. The dipole antennas can be spaced sufficiently to allow a person to be placed within the array. The radio frequency energy from the array can be targeted to a specific location within the array. In the example shown in FIG. 2b, the 3-D cylindrical dipole array consists of a plurality of groups 42 of antennas 44. Each group of antennas 42 includes at least three antennas 34 stacked end to end along the direction of the E-field polarization axis.

Numerical modeling studies show that little improvement is obtained when the number of antenna groups exceeds eight. As the number of groups in the cylindrical shape decreases to fewer than eight, the depth of penetration begins to decrease. Nevertheless, the invention is not limited to eight antenna groups and any number of groups above or below eight is to be considered to be within the scope of the present invention.

It will be appreciated that the three stacked dipoles can be extended to include more dipoles and the phase of each can be controlled to provide proper phase alignment for target selection. If desired in connection with very complex arrays, a computer can be programmed in the controller to select the proper phase and power for each antenna. For purposes of description and not by way of limitation, dipole type antennas are used throughout the description. However, the types of antennas may include patch, metal strips, metallic waveguide, dielectric waveguides, resonant cavities, coaxial antennas, and TEM mode horn type antennas to name but a few types which can be used in practicing the invention.

FIG. 2c illustrates one exemplary embodiment of the three dimensional array 200 of dipole antennas shown in FIG. 2b that is implemented in a system used for hyperthermia treatments. In this example embodiment, the dipole antennas 42 in the array are integrated into a housing 54 in which a person can be placed for hyperthermia treatment. The dipole groups 42 formed normally along an inside wall of the housing. The housing can be constructed of a clear plastic or dielectric material, with the antennas attached using well known adhesives, metal deposition processes, and the like. A thin patch of dielectric coating material 56 can be used to cover the antennae groups 42.

In the example illustrated in FIG. 2c, four circuits are used to feed an electromagnetic signal 47 to the antennas 42. Each circuit is comprised of a phase shifter module 48 that outputs a phase shifted signal 16 to an amplifier 50. The amplified signal is sent through a power divider 52 to separate connector assemblies 36 and to the dipole antennas 42. The phase shifter module can be used to adjust the phase of the signal 47 relative to the signals in the other circuits. The connector assemblies 36 can then be used to further adjust the phase of each signal. For example, a different length of cable can be used between the two antennas 42 and their respective connector assemblies. In addition, open or short connections in the connector assemblies can be used to alter the phase of the signal to provide a desired phase relationship between the radio frequency signals output from the eight dipole groups 42. The system for hyperthermia treatments is more fully described in U.S. Pat. 5,097,844, which is herein incorporated by reference.

The ability to change the phase relationship of the radio frequency signals emitted by the dipole antennas 42 enables the location of maximum power from the radio frequency signals to be steered within the housing 54, thereby enabling effective hyperthermia treatment to be delivered at a specific location. For example, a cross section of a torso 62 is shown inside of a bolus 60 located within the housing. A deposit 41, such as a cancerous tumor, is also shown. The phases of the radio frequency signals can be adjusted to provide constructive interference of the waves near the location of the deposit to maximize the energy of the waves. The frequency of the radio waves can also be selected to provide sufficient penetration into the body to reach the tumor. For example, a frequency of 120 MHz may be used for hyperthermia treatment of tumors located in the torso area. Tumors located in the torso area include tumors of the liver, lung, cervix, pancreas, prostate, or other types of large tumors. A frequency of 915 MHz may be used for hyperthermia treatment of tumors closer to the surface off the body, such as tumors located in the breast, or certain areas of the lungs.

As previously discussed, resonant heating can be induced in deposits of a specific size range. The range in size at which resonant heating can occur within a deposit is dependent on the frequency of the RF radiation that is emitted from the antennas. Generally, resonance can occur in larger deposits at lower frequencies. Higher frequencies, such as the 915 MHz that has been discussed, can be used to provide resonant heating in tumors having a range of approximately 3 to 6 cm. Higher frequencies can also be steered and directed more accurately due to the smaller wavelength of the RF radiation at the higher frequencies. The more accurate steering enables the hyperthermic heating to be more easily contained to the area within the deposit, thereby limiting adverse effects to surrounding tissue.

Unfortunately, in most countries a doctor or treatment administrator typically cannot select a frequency of the RF radiation that would provide optimal resonant heating for a selected deposit size. This is due to the tight control that most countries administer over the radio frequency communications band. Typical frequencies that are allowed include the 915 MHz and 120 MHz frequencies that have been discussed. There is a broad range of deposit or tumor sizes that may not be optimally heated using resonant radio frequency heating at one of these frequencies.

To overcome this limitation, it has been discovered that the dielectric and conductivity properties of the organic tissues comprising the deposit can be altered with injection enhancement of selected materials. For example, saline solution and/or nanoparticles, such as nanotubes, nanospheres, nano balls, and nano rods can be injected into the deposit to alter the dielectric and conductivity properties of the organic tissues comprising the deposit. The saline solution can have a sodium chloride concentration selected to provide a desired increase in the conductivity and dielectric properties of the deposit. The concentration of the saline solution can be selected to be within safe parameters to an individual, while still enabling a desired increase in conductivity and dielectric values. The nanoparticles can be formed of a material such as carbon, gold, platinum, and silver. Other shapes of nanoparticles or types of materials from which they are constructed can also be used to alter the electrical and magnetic properties of the deposit.

By altering the dielectric and conductivity properties of the deposit, the inventors have found that the relative specific absorption rate (SAR) of the RF radiation in the deposit relative to the surrounding tissue can be altered in such a way that the relative SAR can be increased, thereby enabling greater heating of the tumor relative to the surrounding tissue. In one embodiment, the dielectric and conductivity properties of the deposit can be increased to enable the peak relative SAR value to be located at approximately the radius of the deposit, thereby maximizing the heating of the deposit relative to the surrounding tissue.

At radio frequencies and microwave frequencies, the cell wall in human tissue serves as a capacitive as well as a resistive barrier that increases the dielectric constant and decreases electrical conductivity of the tissue as the frequency is reduced. As the frequency increases, the conductivity of the tissue increases due to the reduced capacitive impedance of the cell wall membrane. As the frequency increases, the dielectric constant of tissue drops due to the reduced capacitive impedance of the tissues. Water is the dominant material in human tissues and has a dielectric constant of about 78 in the radio frequency and microwave frequency ranges. Human tissue that is high in water content typically has a dielectric constant of about 45 to 55 at a frequency of 915 MHz. If a nanoparticle, such as a nanotube for example, was able to penetrate through the cell wall membrane and reside at that position, the conductive character of the tissue will increase and the dielectric would also increase as this would provide a more direct pathway through the membrane wall of the cells. The pathway results in a dielectric constant closer to that of water. Such a mechanism can result in a near doubling of the dielectric constant of the tissue and a doubling of the conductivity.

An electromagnetic wave radiated into the tissue has its energy oscillating between electrical and magnetic energy, as can be appreciated. As the electric wave decreases, the magnetic wave increases, and vice versa. It is known that the tissues of the body are not substantially magnetic. Therefore, there is little heat transfer from the magnetic portion of the electromagnetic radiation caused by the magnetic fluctuation of the waves. There is a weaker heating effect where there are induced eddy currents in the conductive tissues that do produce heat. This is due to the permeability of the body tissues being about the same as air. However, when ferromagnetic particles such as nanotubes are placed in these tissues, the ferromagnetic particles can undergo direct heating in the presence of a strong magnetic field. Therefore, if the nanoparticles introduced into the body are of a ferromagnetic nature, there is expected to be some direct energy absorption of the magnetic energy by the ferromagnetic nano particles. The combination of the ferromagnetic heating of such particles combined with the resonance of the RF radiation within the tumor may further enhance the selective heating of a tumor when exposed to an electromagnetic field.

FIGS. 3a-3j are used to show the resonant selective heating that will occur in a spherical tissue mass that is surrounded by another uniform tissue mass, with the spherical tissue mass having various conductivity and dielectric values. These conductivity and dielectric values of the spherical mass may be artificially increased by injecting the mass, or the body, with a fluid configured to increase the conductivity and/or dielectric values of a mass such as a tumor. The fluid may contain sodium chloride, conductive nanoparticles, or other types of materials that can be used to increase the conductivity and dielectric values. The materials are selected to enable the tumor's conductivity and dielectric values to be enhanced, while still providing for the safety of the patient, as previously discussed. Optimally, the injected material will have a desired concentration substantially throughout the mass, with a minimal concentration outside the mass area. Altering the conductivity and dielectric values of the spherical mass enables the SAR of the spherical mass (deposit) to be increased relative to the surrounding tissue.

The specific absorption rate (SAR) of the RF radiation in the spherical tissue mass is proportional to the amount of power being absorbed by the tissues being heated and is also directly proportional to the initial rate of temperature rise in these tissues. The absolute SAR is in units of watts of energy absorbed per kilogram. In such uses of absolute SAR, the value of SAR is directly proportional to the amount of power being applied. So if power is increased by a factor of three, the SAR and heating rate also increases by a factor of approximately three.

It is often useful to evaluate the selective heating of a tumor target mass by the SAR value relative to the surrounding normal tissue. This technique provides a simple way to determine the amount of energy and the heating rate of one tissue type with another. This can be important since hyperthermia treatments work best when a tumor can be selectively heated to a much higher temperature than the surrounding tissue, thereby enabling the tumor to be heated to a level that will reduce or destroy the tumor cells, while causing minimal damage to surrounding healthy tissues.

FIGS. 3a-3j show both the basis and dependence of such resonance phenomena on normal tissues such as a female breast that contains a typical tumor mass. The ratio of the SAR of the tumor mass relative to the SAR of the surrounding tissue provides a method to evaluate the results expected when the conductivity and dielectric constant values of the tumor mass have been artificially increased by injecting conductive material such as saline solution and/or nanoparticles into the tumor mass. A combination of saline solution and nanoparticles may provide the greatest increase in conductivity and dielectric constant values.

The plots shown in FIGS. 3a-3j were generated using a standard and accepted numerical modeling method called MEI theory. This method is rather simple in that it does not model all of the details of a body part, such as a breast, in terms of form and shape, but is based on the assumption that the electromagnetic fields are directed into a tissue zone dominated by fatty tissue, with a tissue sphere (tumor). The relative SAR is compared in a plot with the SAR of the fatty tissue had the tumor model not been present at that location. This provides a numerical technique for predicting the selective heating in the tumor that would result at various frequencies and the resonance that can occur in tumors of various radius sizes.

FIG. 3a shows curves for relative SAR calculations at a frequency of 915 MHz for typical mammary tissue such as a female breast which contains in it a spherical tumor. The plot shows the results over a range of radii. The tumor is modeled to have approximately the same conductivity and dielectric values as muscle. The y axis of the plot is the ratio of the SAR in the spherical tumor relative to the SAR in the mammary tissue. A SAR value that is greater than one infers that there is greater heating in the spherical tumor due to resonance. The x axis shows the calculated SAR ratio for tumors having a radius between zero and ten centimeters.

The solid curve 302 in FIG. 3a shows that the combination of both increased dielectric and conductivity in the breast tumor at the 915 MHz operating frequency provides a significant increase in the specific absorption rate in the tumor tissue compared to the surrounding fatty tissue typical of a breast. The selective resonance of such a typical tumor in a female breast, when exposed to the 915 MHz RF radiation energy, is shown to be enhanced for a tumor radius between 1 and 10 cm. The region where the relative SAR is predicted to triple is for a radius between 1.5 and 4.5 cm. This corresponds to a tumor diameter of between 3 and 9 cm. An even greater enhancement of five times the relative SAR is predicted for a tumor radius of 1.7 and 3.4 cm, or a diameter of 3.4 to 6.8 cm. This size is the common size for an advanced breast cancer tumor.

If the relative SAR is five times greater in the tumor than that of the surrounding tissue due to the resonance of the RF radiation in the tumor, then the initial heating rate would be five times greater than in the surrounding fatty tissue. Such a result would predict that an area of the breast heated in this way may have non-destructive heating of the normal breast tissues, while producing destructive heating in the tumor tissue. Non destructive heating is generally considered to be a temperature that is less than 42 degrees Celsius. Normal body temperature is about 37 degrees Celsius. Therefore, to minimize destructive heating of the breast tissue, it should not be heated more than 5 degrees Celsius. However, with the relative SAR of five times, in the time it takes to heat the surrounding breast tissue by 5 degrees, the tumor tissue can be heated approximately 25 degrees, from body temperature to 62 degrees Celsius. This would result in the selective, non-invasive thermal ablation of such breast tumors.

However, in practice this amount of heating typically does not occur. There may be more blood flowing in the tumor tissue than in the surrounding fatty tissue. The increase in blood flow can moderate or limit the rate at which the temperature of the tumor can be increased relative to the surrounding tissue. In order to provide a greater relative SAR ratio, the dielectric and/or conductivity of the tumor can be increased.

As a comparison, the dash curve 304 shows a theoretical curve for a tumor having the same conductivity as the surrounding fat. The dot curve 306 shows a theoretical curve for a tumor having the same dielectric as the surrounding fat. It can be seen that the heating of the tumor mass relative to the surrounding fat is benefited at smaller tumor sizes by a high conductivity and a low dielectric constant, as shown with the dot curve 306. The dash curve shows there is little benefit of having a relatively high dielectric and a low conductivity. However, the highest relative SAR is obtained with a relatively high conductivity and a high dielectric, as shown with the solid curve 302.

FIG. 3b shows the predicted relative SAR values for a tumor mass heated at a frequency of 915 MHz which has been artificially altered by an agent that changes the dielectric and conductivity uniformly. One cause of an increase in dielectric and conductivity values in a tumor is through an increase in saline fluid and blood. The heavy dash curve 308 is modeled to have a conductivity and dielectric value of such a tumor. The relative SAR value for this tumor has a peak value of about 8 for a spherical tumor that has a radius of about 2.2 cm. The heavy solid line 310, with a dielectric value εᵣ=25 and a conductivity σ=0.24 S/m results in a maximum relative SAR value of only 4.2, which would be less than an untreated tumor, as shown with the solid curve 302 in FIG. 3a.

The remaining plots shown in FIG. 3b illustrate that as the dielectric and conductivity are further increased, the resonance peak sizes increase in amplitude, decrease in the radius of the tumor at which the maximum SAR value occurs, and decrease in effective width. This demonstrates that the characteristics of a tumor can be suitably altered in a stable form for a sufficient period of time to create a selective and destructive temperature rise in the tumor that can enable rapid selective tumor destruction.

In addition, for smaller tumors, such as tumors with a radius less than 2 cm (diameter less than 4 cm), the conductivity and dielectric values of the tumor can be increased to a predetermined amount to enable a peak relative SAR value to occur at approximately the radius of the tumor. For example, for a breast tumor having a radius of 1 cm, the conductivity and dielectric of the tumor may be increased such that the dielectric value is approximately 250 and the conductivity is about 2.40 S/m, as shown by the light solid curve 307. At these dielectric and conductivity values, a peak relative SAR value of 20 occurs for a deposit having a 1 cm radius. At this relative SAR value, a destructive temperature rise in the tumor can easily be achieved with a low amount of negative effect on the surrounding tissue. For example, the surrounding tissue may be increased approximately 2 degrees from 37 degrees Celsius to 39 degrees Celsius, while the tumor will increase from 37 degrees Celsius to approximately 79 degrees Celsius, causing rapid, destructive heating of the tumor tissue.

A conductivity increase can be achieved by an influx of higher concentration saline fluid into the tumor region. The use of saline solution will typically cause a greater increase in conductivity, with a more moderate increase in dielectric. This greater increase in conductivity relative to dielectric value occurs because the relative dielectric value of the water is approximately 78. In high water body tissues the dielectric at a frequency of 915MHz is typically about 51. So a saline injection to such tissues typically causes an increase in dielectric that is less than a factor of two. This may increase the relative SAR, but can have a limited change in the radius of the tumor at which the resonant frequency occurs. By injecting nanoparticles into the tumor tissue or into the body in a way that will enable the nanoparticles to congregate in the tumor tissue, the conductivity and dielectric constant may be increased to provide an increase in SAR that will enhance the hyperthermic treatment of the tumor. The concentration of nanoparticles in the tumor can be selected to provide a desired relative SAR at the tumor's radius.

FIG. 3c shows the effect of increasing the conductivity and dielectric in tumors or other types of deposits that are located other regions of the body, such as the lungs surrounding a tumor mass. In this case, the lung is modeled to have approximately two thirds of the conductivity and dielectric values of muscle. The curves in FIG. 3c show that irradiating a tumor in the lung area with RF radiation at a frequency of 915 MHz is predicted to have very little resonance when the tumor has a conductivity and dielectric value that is approximately double that of the lungs, as shown with the heavy solid line 312. The only substantial improvement in SAR occurs for tumors having a radius greater than 7 cm. If the conductivity and dielectric values of the tumor are increased to 20 times that of the normal lung tissue, an increase in SAR of greater than 4 times can be achieved for tumors having a radius between about 0.5 cm and 0.8 cm, as shown with the light solid line 314. An even greater increase in SAR occurs with a multiple of 50, as shown with the light dash line 316, with the resonant peak increasing and narrowing compared to models for lower multiples of conductivity and dielectric values.

FIG. 3d shows plots for RF radiation at a frequency of 434 MHz that is used to heat a tumor in the female breast. The heavy dash line 318 shows a predicted resonant increase in SAR of at least three times for a tumor having a radius of between 4 and 5.5 cm, and a doubling of the SAR value for a radius between 3.3 and 9 cm. This affects a larger tumor size relative to the use of RF radiation at a frequency of 915 MHz. However, the lower frequency shown in FIG. 3d does not perform as well for smaller tumors. Since most advanced breast tumors that would be considered for hyperthermic treatment will have a size that is less than 8 cm, the use of the 915 MHz RF radiation may be preferred.

FIG. 3e shows a model of a tumor irradiated with RF radiation at a frequency of 434 MHz with surrounding tissue having a dielectric and conductivity of approximately that of lung tissue (⅔ that of muscle and tumor). Some selective resonance is seen for a five fold increase in the resonance and dielectric tissue parameters, as shown in the heavy dashed line 320. The relative SAR is at least 1.5 times greater for a tumor size between 1.5 cm and 4.5 cm. This however, is not a very significant boost of energy absorption relative to the surrounding tissue. At the frequency of 434 MHz, even significant increases in the dielectric and conductivity values of the tumor result in a maximum relative SAR value of 3.0, as shown with the light dashed line 322. This is largely due to the fact that the higher conductive outer tissue loading decreases the resonance peaks of the tumor mass just as a parallel tuned electrical circuit has its resonant currents and voltages limited by parallel resistive elements.

FIG. 3f shows a model of a tumor irradiated with RF radiation at a frequency of 100 MHz in a media having ⅔^{rds} the conductivity and dielectric values of muscle, such as lung tissue. The model shows that some selective tumor heating will occur. For example, the model shows that a tumor having a dielectric value of approximately 100 and a conductivity of 1.0, shown with the light dot curve 324, provides an increase in relative SAR to 1.3 for tumors having a radius of greater than 7 cm. In addition, a tumor having a low dielectric value of approximately 1, shown by the heavy solid curve 326, would have a relative SAR rate of greater than 1.3 for a radius of less than 4 cm. However, it is not expected that tumors will have this lower dielectric value.

FIG. 3g also shows a model of a tumor in lung tissue, as in FIG. 3f. Selective resonant heating occurs with significant increases in the tumor dielectric properties. But only modest increases in the SAR are obtained with reasonable increases in dielectric properties.

FIG. 3h shows the effect of only changing the conductivity of the tumor mass for a typical lung tumor. The benefit is rather limited and provides no selective resonance. The plots indicate that the resonance effects are dependent more on the change in dielectric value than on the conductivity difference. The conductivity increases provides an increase in tumor SAR, but becomes limited at rather high electrical conductivities. The heavy dot line 328, with a conductivity of 5.0 Siemens per meter (S/m) does not provide substantial improvement over the heavy solid line 330 with a conductivity of 1.0 S/m.

FIG. 3i shows that the resonance of an RF radiation signal having a frequency of 100 MHz in fatty tissue, such as the breast. In order to obtain a relative SAR value greater than 1.0, signifying greater heating taking place in a tumor, the tumor size has to be greater than about 7 cm. This is typically larger than most breast tumors. FIG. 3j shows additional plots at 100 MHz. Thus, the use of the 100 MHz frequency is not expected to provide any significant increase in SAR in tumor tissues, even with dielectric manipulation of the tumor, when the tumor is surrounded by fatty tissues such as female breast tissue.

FIGS. 3a-3i show that resonant heating in a tumor can be altered and enhanced in certain situations by increasing the conductivity and/or the dielectric constant of the tumor. Increasing these values is particularly beneficial in tissue regions that have a relatively low dielectric and conductivity. Such tissues are the drier tissues such as fat and bone. However, both lung and brain tissues are also drier than most other body tissues, thereby enabling an amplification in SAR relative to the surrounding tissue by increasing a tumor's conductivity and dielectric values relative to the surrounding tissue.

In another embodiment, FIG. 4 illustrates a simplified hyperthermia system having an array of electromagnetic radiation applicators 14 that can be integrated with a magnetic resonance imaging (MRI) system. The MRI system can be used to image a location of the saline solution or nanoparticles injected into the body, or more specifically, into the tumor. As the conductivity and/or dielectric values of the tumor are artificially increased with the addition of injected material, the MRI system can be used to actively determine a conductivity and/or dielectric value of the tumor. This enables the conductivity and dielectric values to be increased by a selected amount to provide a substantially maximum relative SAR at the size of the tumor, as previously discussed. In addition, the MRI can be used to provide additional heating of the nanoparticles located within the tumor using magnetic waves, as previously discussed.

In one embodiment, the MRI system's electromagnetic radiation (EMR) source 91, can be integrated with the phased array of antennas to provide power for the antennas. The MRI system can also be used to form a temperature image. A user can determine temperature changes caused by the hyperthermia system by taking a plurality of temperature images with the MRI system. An MR temperature image may be taken on a periodic basis, such as every ten minutes. It typically takes approximately one minute to obtain the MR image. Thus, when using the hyperthermia system and the MRI system together, the EMR source 91 can be connected to the hyperthermia system applicators 14 a majority of the time.

When the MRI EMR source 91 is connected to the hyperthermia system applicators 14 the signal path can begin at the MRI EMR source, through a transmission means such as a coaxial cable 98, strip line, or other transmission means, into the switch 99, through another transmission line 93, and into the splitter 11. The MRI EMR source may include a signal generator, power amplifiers, and filters used to provide a substantially noise free signal at a desired frequency. The signal can then be passively spilt at the splitter 11 and directed through transmission line 12 to the central energy supply connection points or feed points 27 of each applicator 14 through cables 17. At these feed points 27, there are additional coaxial cables 19 that are used to attach to variable reflective termination devices 15. These variable reflective termination devices control the effective termination seen at the central energy supply connection points 27 to control the phase at each of the applicators, thereby enabling the heating location within the MRI system to be controlled, as previously discussed. A system for hyperthermia treatments that is incorporated with an MRI system is more fully described in U.S. patent application Ser. No. 12/154,808, filed on Sep. 23, 2008, which is herein incorporated by reference.

One exemplary device which uses an integrated phased array antenna system with a magnetic resonance imaging system is the BSD-2000 made by BSD medical. The system can be used for simultaneous imaging of the heating in the body during such hyperthermia treatment and thermal therapies. Injection of saline solution and/or nanoparticles can also be capable of enhancement of MR imaging to track the distribution of the nanoparticles that become implanted in the target tumor or deposit. This can be used to provide treatment guidance and effectiveness by such targeted treatments. The nanoparticles may also be designed to contain agents such as chemotherapy drugs to further enhance the lethal damage to diseased cells such as malignant tumor cells.

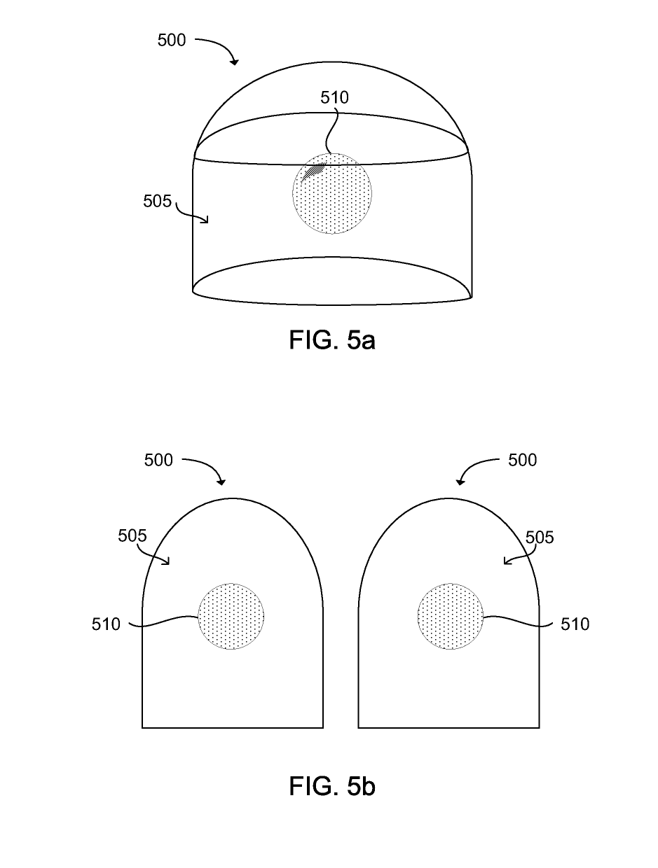

FIG. 5a shows a test phantom material 500 that was configured to simulate a breast formed substantially of fatty breast tissue 505 with a tumor 510 substantially centered therein. A phantom breast can be formed from a variety of different materials that are used to simulate the properties of the fatty breast tissue and a tumor having higher water content. Materials are selected based on their similar properties to breast tissue in the absorption and reflection of radio frequency waves at a selected frequency, such as 915 MHz.

A plurality of different phantom breasts made of various substances were formed and tested using a test device that was configured substantially as illustrated in FIG. 2. One test phantom 500 was formed having an outer area 505 formed of paraffin wax mixed with 0.04% carbon by weight for a dielectric of 8.5 and a conductivity of 0.097 S/m. The wavelength of radio frequency waves at a frequency of 915 MHz in the outer paraffin area is about 11 cm. This wavelength divided by π is about 3.6 cm. The inner area 510 was formed of a saline water based TX-150 gel material to simulate the makeup, electrical, and physical properties of a tumor.

Another test phantom 500 was formed using 1032 grams of wheat flour (64.5%), 464 grams of corn oil (29.9%), 4.1 grams of sodium chloride (0.256%), and 99 grams of water (6.2%) to form the outer area 505. The tumor phantom model 510 was comprised of 89.97% water, 9.8% TX-150, and 0.23% NaCl. The relative permittivity of the fat phantom is approximately 8 to 9. The Lagendijk published conductivity is 0.04 S/m.

The phantom material, as shown in FIG. 5b, can be split in two so that it can be heated and then immediately opened for test and measurement. This is done to provide an accurate measurement of an internal temperature of the simulated fatty tissue 505 and cancerous tumor 510 within the phantom breast 500.

The wax phantom and the flour phantom were both tested under a variety of conditions. In one test of the wax phantom, four power channels that were respectively coupled to the four dipole antennas 29, 31, 33 and 35, as illustrated in FIG. 2. The wax phantom was placed into the aperture 26. The four power channels were set at a relative phase of zero degrees for dipoles 29 and 35 and ninety degrees for dipoles 31 and 33, resulting in a circularly polarized field, as previously discussed. The tests were done with a bolus medium comprised of either deionized water, mineral oil, or an equivalent. The entire volume 24 of the test device can be filled with the bolus medium. Alternatively, the fluid can be confined to an area around the surface of the breast 32. In one test, the split in the phantom was oriented to be centered on dipoles 29 and 35 to allow maximum heating from the dipoles. The two halves of the phantom were separated by a thin sheet of plastic (saran wrap) to reduce evaporative cooling when the phantom was split. A similar setup was used in testing the flour phantom.

The output of the dipole antennas 29, 31, 33 and 35 can be tuned based on the type of bolus used. For example, for an oil bolus the antennas were tuned to be substantially impedance matched with the oil medium. A tuning circuit was adjusted using a Bazooka balun. A Bazooka balun is a quarter wave length of coaxial line that has the outer conductor cut away along a strip on opposite sides forming a quarter wave length of parallel line and at the tip having the center conductor short to one of the outer conductor sides to form one of the two active connection points for a balanced line. Doing this, the impedance match at 915 MHz was between 10 dB to 30 dB return loss. The tuning match was achieved by adding a capacitive shunt at a feed point of the tuning circuit.

Through testing, it was determined that use of an oil bolus with a dielectric between 2.5 and approximately 4 provided less heat absorption by the bolus compared to the use of the deionized water bolus. The lower dielectric value of the oil bolus compared with the deionized water bolus substantially reduces higher order energy modes from propagating in the bolus space. However, a water bolus may still be used if additional methods are used to prevent the higher order modes in the water. Reducing the higher order modes in the water may be accomplished using artificial dielectric modification methods such as low dielectric vanes or sheets that are cross polarized with the dipoles to present perpendicular dielectric boundaries to the electric fields in the radio frequency electromagnetic fields used to selectively heat a tumor. Higher fill factors of the breast relative to the bolus size can also reduce higher order modes. The fluid in the bolus can be circulated to provide additional cooling to the surface of the breast with which the bolus is in contact.

Similar testing to that described above and shown in FIGS. 5a and 5b can be used to verify the predictions made in the plots of FIGS. 3a-3j. For example, saline solution and/or nanoparticles can be infused in the tumor 510 to provide desired dielectric and conductivity values.

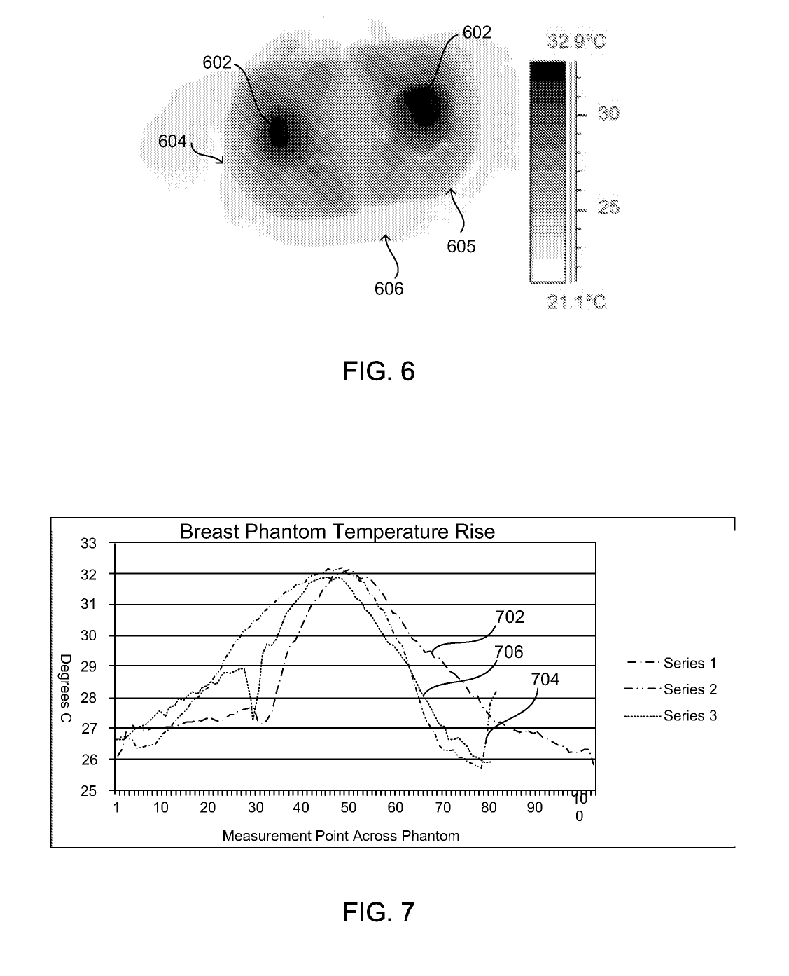

An infrared camera was used to record the temperature of the split phantom after it was selectively heated as previously described. The infrared image shown in FIG. 6 shows the tumor center 602, with no increase in conductivity or dielectric due to an injection of saline solution or nanoparticles, still increased in temperature from a room temperature of about 24 degrees Celsius to a temperature of 32.2 degrees Celsius for a change in temperature of 8.2 degrees Celsius. The phantom surface 604 increased from room temperature to about 27 degrees Celsius. The oil bolus fluid 606 was recorded with a maximum temperature of approximately 25.1 degrees Celsius. The image shown in FIG. 6 shows selective heating of a simulated tumor 602 in a breast equivalent phantom.

The actual amount of power that is sent to each antenna and the length of the exposure can be controlled to achieve desired results. For hyperthermia and thermal therapy systems it is common to monitor target tissue temperature during heating by invasive and at times non-invasive thermometry to provide a control parameter for power levels. In some cases, lower power can be applied for a longer period. For example, 30 watts of power may be sent to each antenna for a period of six or more minutes. Power can be reduced as needed to maintain the desired temperature for a prescribed period that may be as long as 60 minutes. In other cases, it may be desirable to apply more than 50 watts per channel for a relatively shorter period. For example, 100 watts of power may be sent to each antenna for a period of less than two minutes to reach a therapeutic temperature level which will vary with differing blood-flow. In other embodiments, different amounts of power may be sent to each antenna in the array. For example, when a tumor is not centered within the breast, it may be desirable to provide different power ratios to provide substantially even heating on each side of the tumor. The ability to monitor temperature of such a tumor and control the power to maintain a target temperature level is a common element in such applications. Typically temperature is set to over 40 degrees Celsius and maintained for up to 60 minutes. Higher temperatures such as 60 degrees Celsius need only be maintained for less than a few minutes.

FIG. 7 illustrates a diagram showing a plot of the temperature across the breast phantom. The tumor model did not include artificially increased conductivity or dielectric values. It is expected, using the models of FIGS. 3a-3j, that the relative SAR of the tumor 505 can be increased by injecting materials such as saline solution and/or nanoparticles into the tumor model to increase the tumor's conductivity and dielectric values. Increasing the conductivity and dielectric values can also be used to more effectively treat smaller tumors that may not resonate sufficient to increase the relative SAR value to a point where destructive heating can occur within the tumor.

The Series 1 line 702 represents 100 temperature points taken across the wax phantom from left to right passing through the center of the tumor model. The Series 2 line 704 represents 100 temperature points taken across the phantom from back to front through the center of the tumor model. The Series 3 line 706 represents 100 temperature points taken in a diagonal from the left base of the breast phantom passing through the central tumor model and ending at the frontal side surface of the wax breast phantom. Each of the series lines show that the temperature dramatically increases at a center of the phantom, where the tumor model is located, thereby showing selective heating of a tumor in the breast equivalent phantom.

Additional testing was performed, with each test showing a significant heating of the tumor relative to the surrounding tissue. The following table summarizes the temperature change of the tumor material in the paraffin phantom and the surface temperature of the phantom as a result of short term heating experiments to measure where the power is being primarily absorbed.

| Tests | Tumor Max Temp Change | Surface Max Temp Change |
|---|---|---|
| 1 | 8.2° C. | 3.0° C. |
| 2 | 11.8° C. | 3.9° C. |
| 3 | 10.7° C. | 5.4° C. |
| 4 | 8.8° C. | 2.7° C. |

The average ratio from these tests of the tumor maximum temperature rise versus the average maximum temperature rise on the phantom surface is 9.875/3.75=2.63. Thus, a tumor located in a fatty breast tissue can be selectively heated using radio frequency waves when the tumor has a diameter that is around ⅓^{rd} of the wavelength of a radio frequency electromagnetic field in the fatty tissue. In other words, the wavelength of the radio frequency waves in the fatty breast tissue will be about three times a width of the tumor for optimal resonant heating. Of course, resonant heating can still be attained within a range of wavelengths. The wavelength of the radio frequency waves in the fatty tissue may be ±about 25% of the optimal length, or in other words about 3.75 times a diameter of the tumor to about 2.25 times a diameter of the tumor. Alternatively, a single frequency such as 915 MHz can be used to provide resonant heating of tumors having a diameter of about 2.3 cm to over 7 cm, with an increased amount of selective heating when the tumor has a size of about 4.5 cm.

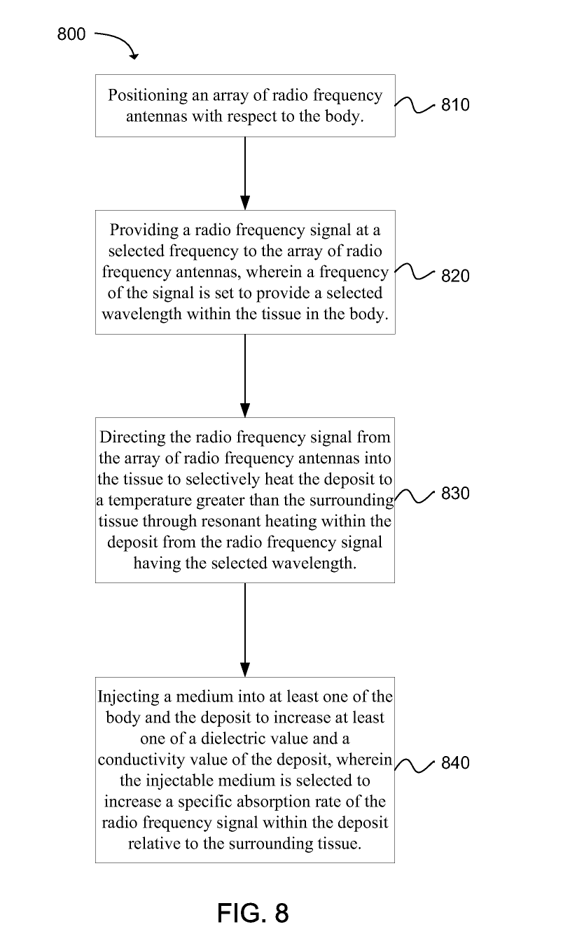

In another embodiment, a method 800 of selectively heating a deposit surrounded by tissue in a body is disclosed, as depicted in the flow chart of FIG. 8. The method comprises positioning 810 an array of radio frequency antennas with respect to the body. A radio frequency signal is provided 820 at a selected frequency to the array of radio frequency antennas. The frequency of the signal is set to provide a selected wavelength within the tissue in the body. The radio frequency signal is directed 830 from the array of radio frequency antennas into the tissue to selectively heat the deposit to a temperature greater than the surrounding tissue through resonant heating within the deposit from the radio frequency signal having the selected wavelength. A medium is injected 840 into at least one of the body and the deposit to increase at least one of a dielectric value and a conductivity value of the deposit. The injectable medium is selected to increase a specific absorption rate of the radio frequency signal within the deposit relative to the surrounding tissue. The injectable medium can include saline solution and/or nanoparticles to increase at least one of a dielectric value and a conductivity value of the deposit. The injectable medium can be injected to provide a desired concentration of the medium within the deposit to increase the value of the relative SAR. In one embodiment, the desired concentration can be selected to provide a substantially maximum relative SAR at the deposit's radius through resonant heating of the deposit with the radio frequency signal.

The heating described can be used to selectively heat a deposit in the body such as a tumor to enhance tumor therapeutic affects by other cancer treatments such as radiation or chemotherapy (such as selective heat release of liposome encapsulated chemotherapy), or both. As one non-limiting example of a combined therapy/hyperthermia treatment, a traditional cancer therapy (e. g., chemotherapy and/or radiation) is given to a patient and followed by a computed tomography (CT) scan or other appropriate scanning technique to locate the precise location of the tumor within the tissue. The hyperthermia treatment is then given as described hereinabove. After the final hyperthermia treatment is given, a radiation oncologist can measure the tumor shrinkage by any suitable means, and recommend the least invasive type of surgery to remove the tumor. Surgery is followed by additional therapy and hyperthermia treatment, if one or both procedures are indicated at this stage, to kill any undetected cancer cells in the tissue.

It can be appreciated that the embodiments disclosed hereinabove have potential applications outside the immediate scope of cancer therapy, such as cellular necrosis, chemical reaction kinetics, and catalysis. It will also be understood only examples of the invention have been shown and described and that various details of the invention may be changed without departing from the scope of the invention. Furthermore, the foregoing description is for the purpose of illustration only, and not for the purpose of limitation.

What is claimed is:

1. An apparatus for providing hyperthermia treatments in a deposit surrounded by tissue in a body, comprising:
a first radio frequency antenna operable to direct a radio frequency signal at a selected frequency into the tissue such that the radio frequency signal will have a selected wavelength to selectively heat the deposit to a temperature greater than the surrounding tissue through resonant heating within the deposit from the radio frequency signal; and
an injectable medium operable to be injected into the deposit to increase at least one of a dielectric value and a conductivity value of the deposit, wherein the injectable medium is selected to increase a specific absorption rate of the radio frequency signal within the deposit relative to the surrounding tissue.

2. The apparatus of claim 1, wherein the injectable medium is selected to provide a substantially maximum specific absorption rate of the radio frequency signal within the deposit at about a diameter of the deposit.

3. The apparatus of claim 1, wherein the injectable medium contains at least one of nanoparticles and saline solution.

4. The apparatus of claim 3, wherein the nanoparticles are selected from the group consisting of nano-tubes, nano-spheres, nano-balls, and nano-rods.

5. The apparatus of claim 3, wherein the nanoparticles are formed from a material selected from the group consisting of carbon, gold, platinum, and silver.

6. The apparatus of claim 3, wherein the nanoparticles are formed from a ferromagnetic material.

7. The apparatus of claim 1, further comprising a radio frequency signal generator operable to generate the radio frequency signal at the selected frequency.

8. The apparatus of claim 1, wherein a diameter of the deposit is within a range of about 0.5 times to 0.16 times the wavelength of the radio frequency signal within the tissue

9. The apparatus of claim 1, wherein the deposit is a cancerous tumor.

10. The apparatus of claim 1, wherein the selected frequency of the radio frequency signal is selected from the group consisting of 915 MHz and 120 MHz.

11. The apparatus of claim 1, wherein the deposit has a diameter of in a range of about 1 centimeter to 10 centimeters.

12. The apparatus of claim 1, wherein the deposit has a diameter in a range of about 3.4 to 6.8 centimeters.

13. The apparatus of claim 1, further comprising second, third, and fourth antennas surrounding the deposit and operable to direct a radio frequency signal at the selected frequency into the tissue such that the radio frequency signal will have the selected wavelength to selectively heat the deposit to a temperature greater than the surrounding tissue through resonant heating within the deposit from the radio frequency signal.

14. The apparatus of claim 13, wherein a phase difference between the first and third antennas and the second and fourth antennas is approximately equal to 90 degrees to provide circular polarization of radio frequency electromagnetic radiation within the deposit.

15. The apparatus of claim 13, wherein a phase difference between the first and third antennas and the second and fourth antennas is less than or greater than 90 degrees to provide elliptical polarization of radio frequency electromagnetic radiation within the deposit.

16. The apparatus of claim 1, further comprising a fluid filled bolus positioned in a cavity between the surrounding tissue and the first radio frequency antenna.

17. The apparatus of claim 16, wherein the fluid in the fluid filled bolus is a substantially non-ionic fluid having a dielectric constant less than ten.

18. The apparatus of claim 16, wherein the fluid in the fluid filled bolus is selected from the group consisting of mineral oil, vegetable oil, propylene glycol, ethylene glycol, deionized water, liquid silicon.

19. A method of selectively heating a deposit surrounded by tissue in a body, comprising:
positioning an array of radio frequency antennas with respect to the body;
providing a radio frequency signal at a selected frequency to the array of radio frequency antennas, wherein a frequency of the signal is set to provide a selected wavelength within the tissue in the body;
directing the radio frequency signal from the array of radio frequency antennas into the tissue to selectively heat the deposit to a temperature greater than the surrounding tissue through resonant heating within the deposit from the radio frequency signal having the selected wavelength; and
injecting a medium into at least one of the body and the deposit to increase at least one of a dielectric value and a conductivity value of the deposit, wherein the injectable medium is selected to increase a specific absorption rate of the radio frequency signal within the deposit relative to the surrounding tissue.

20. A method as in claim 19, wherein directing the radio frequency signal further comprises directing the radio frequency signal from the array of radio frequency antennas to provide a circularly polarized electromagnetic field at the selected wavelength within the deposit to provide substantially even resonant heating of the deposit to enable the deposit to be heated to a substantially higher temperature than the surrounding fatty mammary tissue.

21. A method as in claim 19, wherein injecting the medium further comprises injecting a medium containing at least one of saline solution and nanoparticles, wherein the nanoparticles are formed from a material selected from the group consisting of carbon, gold, platinum, and silver.

22. A method as in claim 21, further comprising injecting the medium containing nanoparticles, wherein the nanoparticles have a size selected to enable a portion of the nanoparticles to penetrate a cell wall within the deposit.
